(19) United States
(12) Patent Application Publication
(10) Pub. No.: US 2014/0135730 A1
(43) Pub. Date: May 15, 2014

(54) Adjustable Pant-Like Disposable Undergarment With Fully Severed Front Panel And Method Of Forming Through Two Cutting Steps

(71) Applicant: KIMBERLY-CLARK WORLDWIDE, INC., Neenah, WI (US)

(72) Inventors: Joseph A. Mlinar, Appleton, WI (US); Joseph Daniel Coenen, Kaukauna, WI (US); Jason Andrew Verboomen, Appleton, WI (US); Bradley William Schoon, Oshkosh, WI (US); Brian Keith Rhodes, Larsen, WI (US); Robert Lee Popp, Greenville, WI (US); Steven Alan Kolasinski, New London, WI (US)

(73) Assignee: Kimberly-Clark Worldwide, Inc., Neenah, WI (US)

(21) Appl. No.: 13/677,402

(22) Filed: Nov. 15, 2012

(51) Int. Cl. A61F 13/15; A61F 13/56

(52) U.S. Cl. CPC A61F 13/15585; A61F 13/5633
USPC 604/386; 493/343

(57) ABSTRACT

Generally, a pre-fastened adjustable pant-like disposable absorbent undergarment including a fully severed front body panel and method of manufacturing is disclosed. The method includes the steps of providing a moving web for use as a body panel and first forming a first cut on the moving web at a first cutting station to define a leading portion of the moving web and a trailing portion of the moving web connected at the first cut. The moving web then passes through an attachment assembly station to bridge the connected leading portion and trailing portion together with a fastener assembly that extends over the first cut. Finally, the moving web is then passed through a second cutting station to provide a second cut through both the moving web and fastener assembly and intersecting with the first cut to completely separate the leading portion and trailing portion of the moving web.

### BACKGROUND

Pant-like disposable undergarments for absorbing human discharges can appear similar in size and shape to regular cloth underwear which is designed to be laundered and reused. A disposable absorbent undergarment is intended to be worn by persons, including infants, toddlers, or adults, and is designed for a single or temporary use and is meant to be disposed of after being used once instead of being laundered or dry cleaned for re-use. Some examples of disposable undergarments include infant diapers, training pants, adult incontinence garments, feminine pants, etc.

Some pant-like disposable absorbent undergarments manufactured today resemble regular cloth underwear in that they have a waist opening and a pair of leg openings. Such pant-like disposable absorbent undergarments can be pulled up around the torso of a wearer in a similar fashion as regular cloth underwear. Still other pant-like disposable absorbent undergarments have an open or flat configuration and are designed to be placed adjacent to a wearer's torso and then rely upon one or more attachment tabs or fasteners to secure the undergarment around the wearer's torso. This design is beneficial for bed bound users who may be immobile or for babies or children who need assistance in securing the undergarment in place. Still other adjustable, pant-like absorbent undergarments contain attachment means for opening and closing the waist opening after the undergarment has been positioned around the wearer's torso. This type of adjustable undergarment has an advantage in that the wearer does not have to remove outer clothing in order to check the status of the undergarment or to remove the undergarment from their body.

One example of such an adjustable, pant-like disposable absorbent undergarment includes a pair of lines of weakness that a user must break to enable adjusting the fit of the undergarment. The lines of weakness usually extend from the waist opening to one of the leg openings and are designed to be broken either prior to positioning the undergarment around the user's torso or while the undergarment is already positioned around the wearer's torso. A pair of fastener assemblies or attachment tabs is then utilized to refasten the undergarment so that it is snug about the wearer's torso.

It has been found that a major portion of each of the lines of weakness is visually hidden and some users cannot see them and thereby do not know that they are present. In addition, each line of weakness may be ergonomically hard to tear open by older adults, some of who may be suffering from arthritis or other physical ailments. In addition, options that require tearing often indicate to users that the product is damaged or of poor quality when torn.

An adjustable, pant-like disposable absorbent undergarment that includes a fully severed panel versus one the user need to tear open and more easily accessible fastener mechanisms is needed. Typically, however, the types of consumer goods mentioned above are manufactured on a continuous basis on large scale manufacturing lines. Usually, various raw products or components are formed on, or integrated into, a continuous stream of material, which often includes a web of material that moves in a machine direction through and along the line. As such, it is important to maintain the integrity of the stream of material or web during the process so as to avoid costly downtime. In general, the web is pushed or pulled along the line, so as to put the web in tension. Accordingly, the formation of a fully severed panel, especially along a cross-direction, can increase the risk of web breakage resulting in machine downtime. Therefore, it is desirable to maintain the tensile strength of the stream of materials or web as it passes through the process.

Therefore, there is need to provide a method and apparatus for manufacturing pant-like disposable absorbent undergarment that includes a fully severed panel without causing a problem with the integrity of the stream of material or web during the manufacturing process.

### SUMMARY

Generally, a pre-fastened adjustable pant-like disposable absorbent undergarment including a fully severed front body panel and method of forming such an article is disclosed. The method includes the steps of providing a moving web for use as a body panel and first forming a first cut on the moving web at a first cutting station to define a leading portion of the moving web and a trailing portion of the moving web connected at the first cut. The moving web then passes through an attachment assembly station to bridge the connected leading portion and trailing portion together with a fastener assembly that extends over the first cut. Finally, the moving web is then passed through a second cutting station to provide a second cut through both the moving web and fastener assembly and intersecting with the first cut to completely separate the leading portion and trailing portion of the moving web, such that the leading portion and trailing portion remains bridged together by the fastener assembly.

In another embodiment, the process includes monitoring the attachment of the fastener assembly. If the fastener assembly is properly attached, the second cut is completed. If the fastener assembly is not properly attached, the second cut is not completed.

Preferably, both first cutting station and second cutting station form a series of intermittent cuts in the moving web. In one desirable embodiment, the first cut includes a series of intermittent cuts, each perforation extending in both a machine direction and a cross-machine direction. In this embodiment, the second cut may extend in only the machine direction or cross-machine direction or in both a machine direction and a cross-machine direction as long as the second cut intersects the first cut to provide a full cut across the moving web.

In another embodiment, the first cut comprises a series of intermittent cuts extending in only a cross-machine direction. In this embodiment, the second cut may extend in only the cross-machine direction or in both a machine direction and a cross-machine direction as long as the second cut intersects the first cut to provide a full cut across the moving web.

In other embodiments, the first cut includes at least two lines of intermittent cuts, wherein the two lines of intermittent cuts extend across the entire cross-machine direction of the moving web and extend in at least one overlapping area in the machine direction. In this embodiment, the second cut intersects the two lines of intermittent cuts in the overlapping area to provide a full cut across the moving web.

In other embodiments, the second cut may be a series of cuts removing a portion of the fastener and moving web to form shaped cuts. The shaped cuts may be circles, squares, or any other shape known to one skilled in the art.

In another embodiment, the fastener assembly is provided with at least one hole formed therethrough. In this embodiment, cutting a second cut may be done through the moving web and through the at least one hole in the fastener assembly, the second cut intersects the first cut such that the leading portion of the moving web and trailing portion of the moving web completely separate, wherein the leading portion and trailing portion remains bridged together by the fastener assembly.

In another embodiment, the second cut intersects the first cut such that the leading portion of the moving web and trailing portion of the moving web substantially separate, wherein the leading portion and trailing portion remains bridged together by the fastener assembly. In this embodiment, it is preferred that at least 90% of the moving web between a waist end and a leg cut-out end of the moving web is cut.

Generally, the method described above produces a pre-fastened adjustable pant-like disposable absorbent undergarment having a body panel and a fastener assembly attached to the body panel, wherein at least one side of the fastener assembly is releasably attached to the body panel. The body panel contains a first cut through the body panel under the fastener assembly defining a first section of the body panel and a second section of the body panel. A second cut through both the body panel and fastener assembly intersects the first cut to completely separate the first section of the body panel and the second section of the body panel, wherein the first section and second section remains bridged together by the fastener assembly.

### BRIEF DESCRIPTION

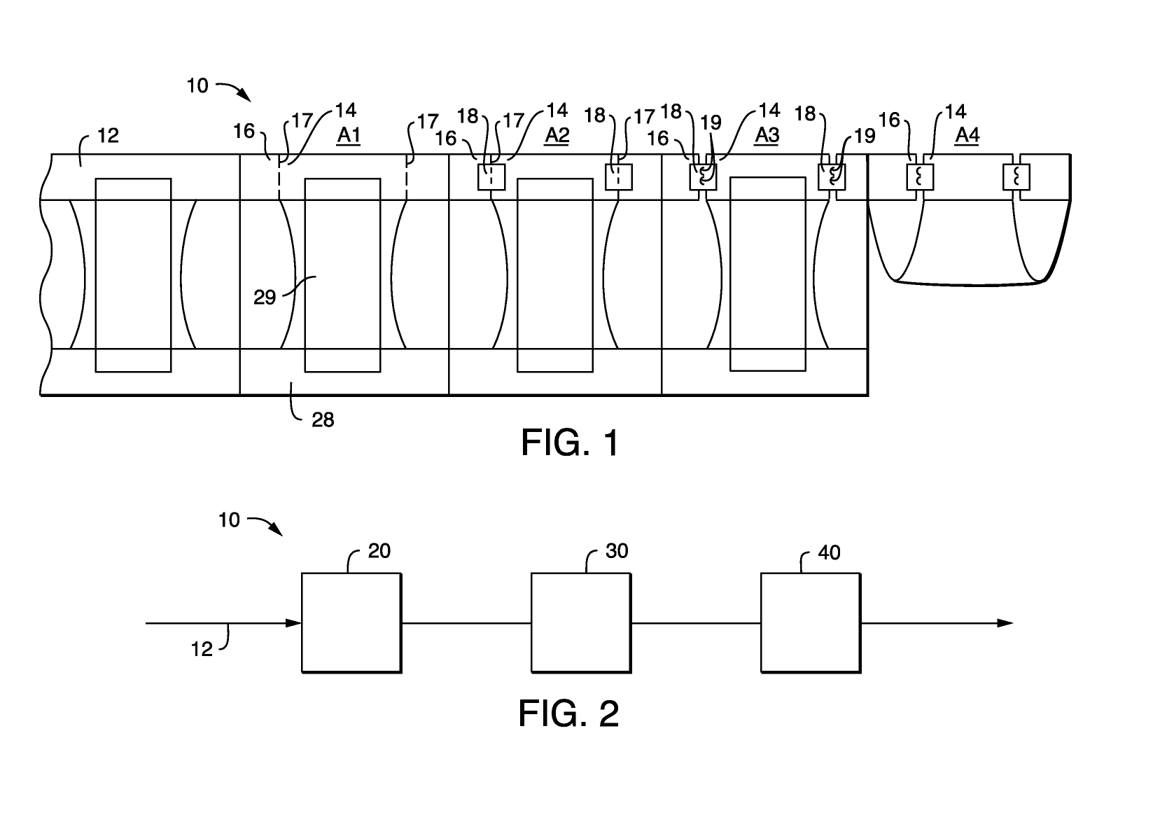

FIG. 1 depicts a method of producing a pre-fastened adjustable pant-like disposable absorbent undergarment including a fully severed front body panel.

FIG. 2 depicts an apparatus for producing a pre-fastened adjustable pant-like disposable absorbent undergarment including a fully severed front body panel.

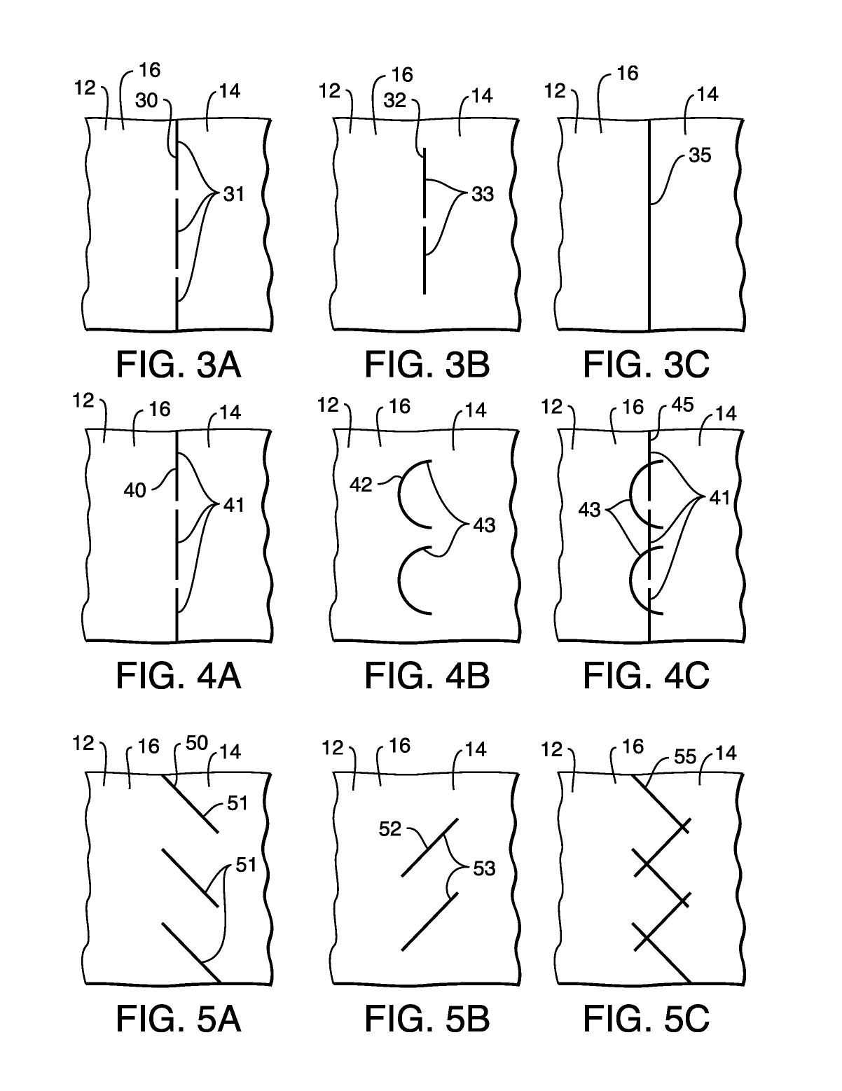

FIG. 3a-3c depict an exemplary embodiment of patterns for a first cut, a second cut, and the two cuts when combined together for use with the method and apparatus of FIGS. 1 and 2.

FIG. 4a-4c depict an alternative embodiment of patterns for a first cut, a second cut, and the two cuts when combined together for use with the method and apparatus of FIGS. 1 and 2.

FIG. 5a-5c depict an alternative embodiment of patterns for a first cut, a second cut, and the two cuts when combined together for use with the method and apparatus of FIGS. 1 and 2.

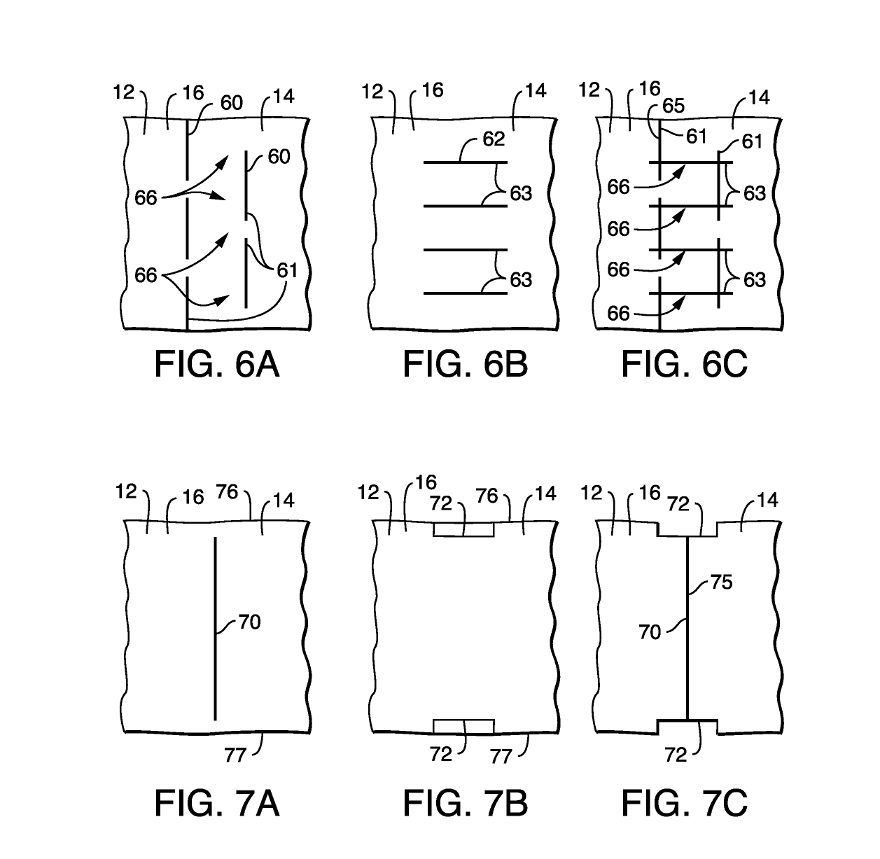

FIG. 6a-6c depict an alternative embodiment of patterns for a first cut, a second cut, and the two cuts when combined together for use with the method and apparatus of FIGS. 1 and 2.

FIG. 7a-7c depict an alternative embodiment of patterns for a first cut, a second cut, and the two cuts when combined together for use with the method and apparatus of FIGS. 1 and 2.

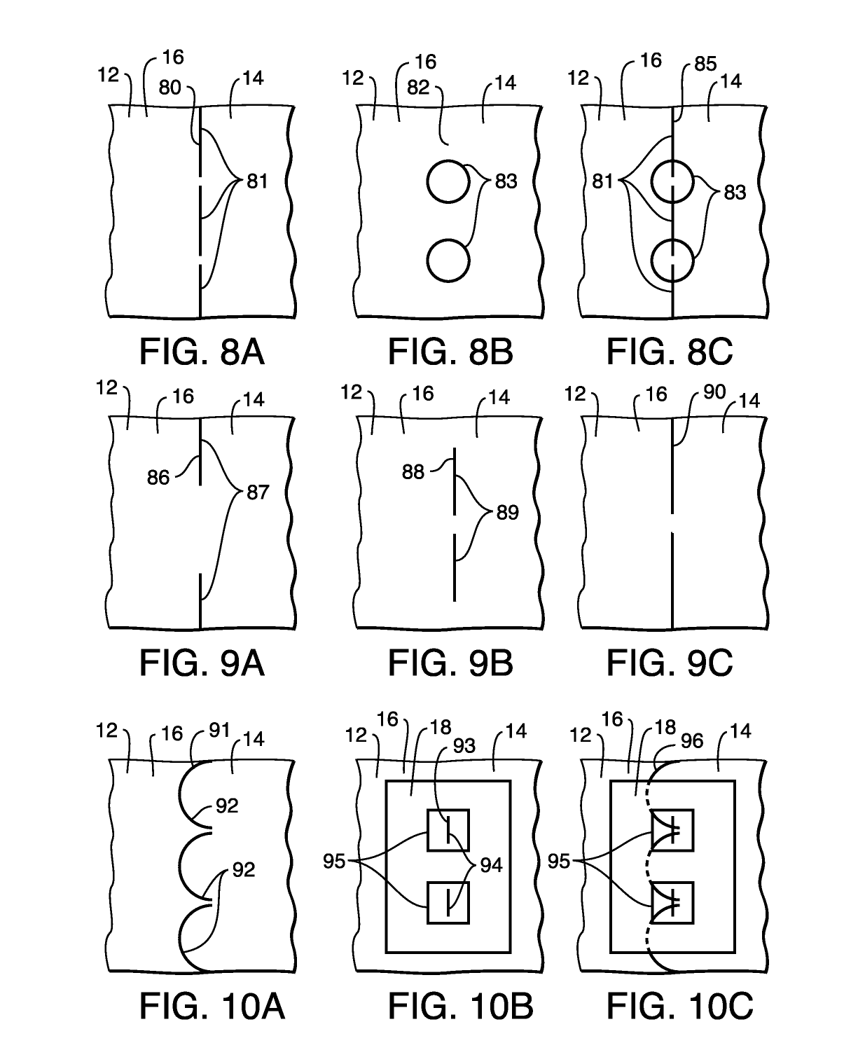

FIG. 8a-8c depict an alternative embodiment of patterns for a first cut, a second cut, and the two cuts when combined together for use with the method and apparatus of FIGS. 1 and 2.

FIG. 9a-9c depict an alternative embodiment of patterns for a first cut, a second cut, and the two cuts when combined together for use with the method and apparatus of FIGS. 1 and 2.

FIG. 10a-10c depict an alternative embodiment of patterns for a first cut, a second cut, and the two cuts when combined together for use with the method and apparatus of FIGS. 1 and 2.

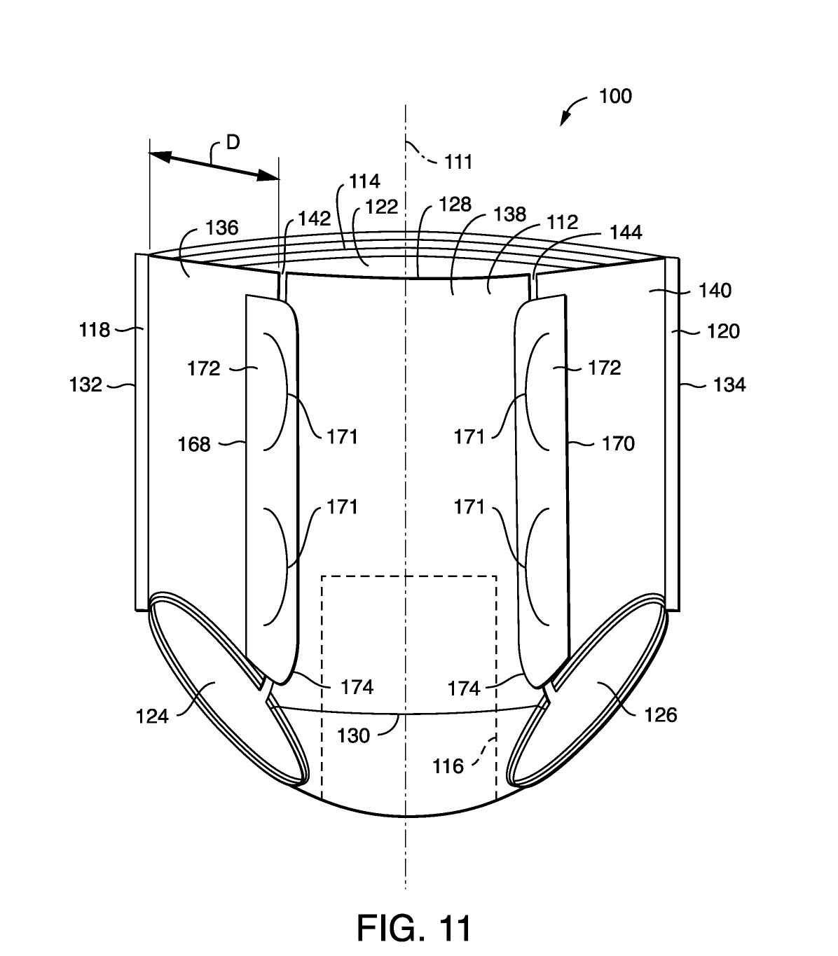

FIG. 11 depicts a front perspective view of one example of a garment, the garment shown in a pre-fastened, pant-like configuration.

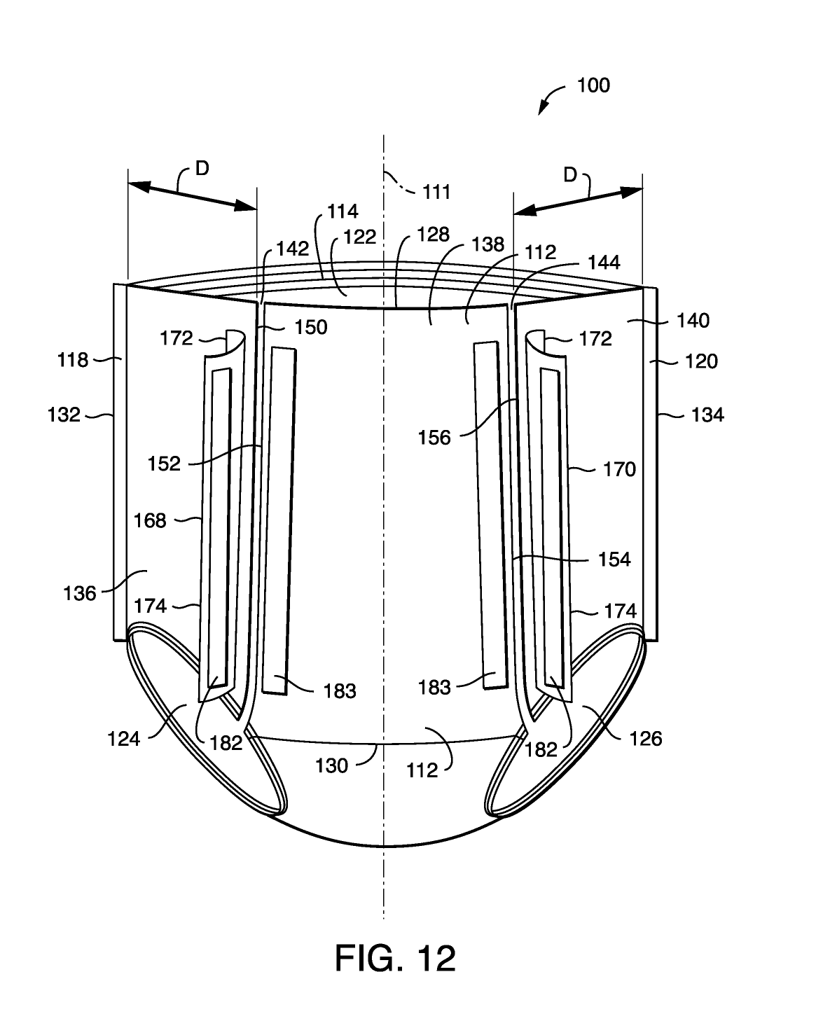

FIG. 12 depicts a front perspective view of one example of a garment, the garment shown in an unfastened, pant-like configuration.

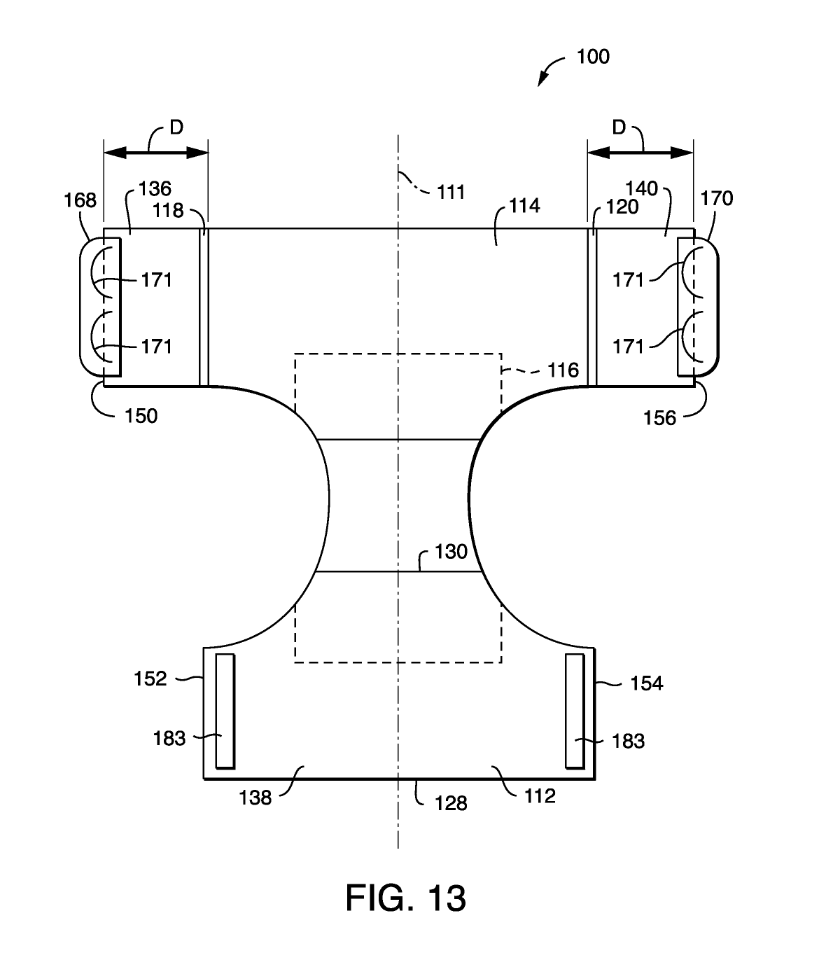

FIG. 13 depicts a plan view of the garment of FIG. 1, the garment shown in an unfastened, laid-open, relaxed configuration.

### DETAILED DESCRIPTION

Reference to the Figures shall be made in describing various embodiments. It should be noted that the embodiments depicted in the Figures and described herein are merely representative examples. The various embodiments are suitable for use in conjunction with disposable absorbent undergarments such as refastenable adult incontinence underwear, pre-fastened disposable diapers, refastenable disposable training pants or swim pants, refastenable disposable enuresis garments, and the like. For illustration purposes, various embodiments shall be described in conjunction with refastenable incontinence or enuresis underwear.

Within the context of this specification, each term or phrase below will include the following meaning or meanings.

The term “body side” should not be interpreted to mean in contact with the body of the user, but rather simply means the side that would face toward the body of the user when the garment is applied to the user, regardless of whether the absorbent garment is actually being worn by the user and regardless of whether there are or may be intervening layers between the component and the body of the user. Likewise, the term “garment side” should not be interpreted to mean in contact with the garments of the user, but rather simply means the side that faces away from the body of the user when the garment is applied to the user, and therefore toward any outer garments that may be worn by the user, regardless of whether the absorbent garment is actually being worn by a user, regardless of whether any such outer garments are actually worn and regardless of whether there may be intervening layers between the component and any outer garment.

The term “machine direction” means the direction of flow as the various members and webs progress along the fabrication line and process. It should be understood that various separate members or webs can each be traveling in a machine direction, but with the various machine directions not necessarily being parallel or oriented in the same direction. For example, one web may be traveling along a first machine direction, which is substantially perpendicular to the travel of another web in a second machine direction.

The term “cross-machine direction” means the direction substantially perpendicular to the machine direction.

The term “downstream” means that one item is positioned more closely to the output or finished product end of the machine and/or process relative to another item. Conversely, the term “upstream” means that an item is positioned more closely to the input end of the machine or process relative to another item. For example, the output end is downstream of the input end, and vice versa, the input end is upstream of the output end.

The term “disposable absorbent undergarment” as used herein is an article that is intended to be worn by persons, including infants, toddlers or adults, which is designed for a single or temporary use and is meant to be disposed of after being used once instead of being laundered or dry cleaned for re-use.

The term “attached” refers to the joining, adhering, bonding, connecting, or the like, of two elements. Two elements will be considered to be attached together when they are attached directly to one another or indirectly to one another, such as when each is directly attached to intermediate elements.

The term “disposable” refers to articles which are designed to be discarded after a limited use rather than being laundered or otherwise restored for re-use.

The term “elastomeric” refers to a material or composite which can be elongated by at least 50% of its relaxed length and which will recover, upon release of the applied force, at least 20% of its elongation. It is generally preferred that the elastomeric material or composite be capable of being elongated by at least 100%, more preferably by at least 200%, of its relaxed length and recover, upon release of an applied force, at least 50% of its elongation.

The term “stretchable” refers to a material or composite which can be elongated by at least 5% of its relaxed length without breaking with or without recovery. It is generally preferred that the stretchable material or composite be capable of being stretchable by at least 10%, more preferably by at least 20% or more, of its relaxed length without breaking.

“Longitudinal” and “transverse” have their customary meaning, as indicated by the longitudinal axis depicted in the Figures. The longitudinal axis lies in the plane of the article and is generally parallel to a vertical plane that bisects a standing wearer into left and right body halves when the article is worn. The transverse axis lies in the plane of the article generally perpendicular to the longitudinal axis.

These terms may be defined with additional language in the remaining portions of the specification.

Generally, a method and apparatus for producing a pre-fastened adjustable pant-like disposable absorbent undergarment including a fully severed front body panel is disclosed. The method includes the steps of providing a moving web for use as a body panel and first forming a first cut on the moving web at a first cutting station to define a leading portion of the moving web and a trailing portion of the moving web connected at the first cut. The moving web then passes through an attachment assembly station to bridge the connected leading portion and trailing portion together with a fastener assembly that extends over the first cut. Finally, the moving web is then passed through a second cutting station to provide a second cut through both the moving web and fastener assembly and intersecting with the first cut to completely separate the leading portion and trailing portion of the moving web, such that the leading portion and trailing portion remains bridged together by the fastener assembly.

Referring to FIGS. 1 and 2, a method and apparatus for producing a pre-fastened adjustable pant-like disposable absorbent undergarment is illustrated. A moving web 12 is shown as moving in a machine direction along a process line. The moving web 12 may be used as a front body panel, a back body panel, or both in the disposable absorbent undergarment.

The moving web 12 can be formed from a single piece of material or can be formed as a laminate consisting of two or more layers. The layers of the laminate can be of the same material or different material. In one embodiment, a laminate is formed from a first layer and a second layer. Sandwiched between the first and second layers are two or more elastic strands. Desirably, from two to about a hundred elastic strands can be utilized in the moving web 12 depending upon the overall size of each panel. The elastic strands can be formed from LYCRA, or a similar material. LYCRA is a trademark of INVISTA (Wichita, Kans.). The diameter and/or cross-sectional configuration of the elastic strands, the decitex (weight in grams per 10,000 meters) of the elastic strands, and the tension imparted into the elastic strands can all be varied to suit a particular product needs. The exact number of elastic strands that are utilized should be sufficient to ensure that the disposable absorbent undergarment 10 snuggly conforms to the wearer's torso.

The elastic strands can be coated with an adhesive. By adhesively coating each of the elastic strands, instead of slot coating a major portion of the inner surface of at least one of the first and second layers, softer moving webs 12 respectively, can be obtained. Wearers of disposable absorbent undergarments prefer a product that has a softer feel since it is more like underwear.

In other embodiments, the elastomeric nonwoven material comprises an elastomeric film sandwiched between two nonwoven facing layers. U.S. Pat. No. 7,803,244 to Siqueira et al., hereby incorporated by reference, discloses particular examples of elastomeric nonwoven composites suitable for use in the disposable adjustable undergarment described herein.

It should be noted that the moving web 12 can be formed from a breathable or a non-breathable material. Desirably, the moving web 12 is formed from a breathable material or a material that is treated or processed to be breathable. Spunbond and bonded carded webs are two breathable materials that work well to form a moving web 12 in disposable absorbent undergarments. Spunbond and bonded carded webs are produced and commercially sold by a variety of vendors. Other materials that can be used to form the moving web 12 include woven and non-woven materials formed from natural or synthetic fibers; polyolefins, such as polypropylene or polyethylene; thermoplastic films; as well as other materials known to those skilled in the art. A metallocene polypropylene works very well since it has a soft feel and can be easily ultrasonically bonded to itself.

The moving web 12 is first passed through a first cutting station 20 at a first position A1. The first cutting station 20 forms a first cut 17 in the moving web 12 to define a leading portion 14 of the moving web 12 and a trailing portion 16 of the moving web 12 remaining connected at the first cut 17. The first cut 17 can include a perforation, die cut or other series of cuts, or breakage or separation of materials.

The first cut 17 may extend across the entire cross-direction width of the moving web 12, or along only a portion thereof. In some embodiments, the first cut 17 can be tapered relative to the longitudinal axis 111, if desired. In addition, the first cut 17 can also be curved. The first cut preferably has a length and which may or may not have a defined width, and can include linear and non-linear patterns, such as curvilinear patterns of cuts, or other shapes, such as a circles, rectangles, etc.

In a desirable embodiment, the first cutting station 20 forms a series of intermittent cuts in the moving web 12. In one desirable embodiment, the first cut includes a series of intermittent cuts, each perforation extending in both a machine direction and a cross-machine direction across the moving web 12. In another embodiment, the first cut 17 includes a series of intermittent cuts extending in only a machine direction or a cross-machine direction across the moving web 12. In other embodiments, the first cut 17 includes at least two lines of intermittent cuts, wherein the two lines of intermittent cuts when combined extend across the entire cross-machine direction of the moving web 12 and extend in at least one overlapping area in the machine direction. The first cut 17 may be any other shape of cut known to those skilled in the art as long as the first cut 17 maintains the web integrity and does not break the moving web 12 between the leading portion 14 and the trailing portion 16 of the moving web 12. A cut extending in the machine direction extends in the transverse direction on the garment while a cut extending in the cross-machine direction extends in the longitudinal direction on the garment.

In one desirable embodiment, the first cutting station 20 is configured as a perforator having a knife roll and an anvil roll. Alternatively, the first cutting station 20 can be configured with a die cutter, laser, water jet, or other types of cutters known to those skilled in the art. In other alternative embodiments, the first cutting station 20 can comprise a device for applying heat, thermal energy or ultrasonic energy to the web so as to create cuts in the moving web at specific locations. In other preferred embodiments, the first cutting station 20 can include a chemical applicator that applies various chemicals, including for example water, to the web to cut the web at specific locations. In yet another alternative embodiment, the apparatus applies a speed differential to the web so as to from cuts the moving web 12. Of course, it should be understood that the first cutting station 20 can also be configured from combinations of one or more of the above-referenced devices.

Downstream from the first cutting station 20, the moving web 12 is passed through a fastener assembly attachment station 30 at a second position A2. The fastener assembly attachment station 30 attaches a fastener assembly 18 that bridges the connected leading portion 14 of the moving web 12 and trailing portion 16 of the moving web 12 together with a fastener assembly 18 that extends over the first cut 17. The fastener assembly 18, which may be applied soon after the first cut 17 is formed, help to maintain the integrity of the moving web 12 as it continues through the process. The fastener can be securely attached using an adhesive, heat, pressure, a combination of heat and pressure, an ultrasonic bond, a chemical bond or by other means known to those skilled in the art. The fastener may also be a refastenable mechanical fastener.

Downstream from the fastener assembly attachment station 30, the moving web 12 is passed through a second cutting station 40 at a third position A3. The second cut 19 intersects with the first cut to completely separate the leading portion 14 of the moving web 12 and trailing portion 16 of the moving web 12 at the first cut 17. In other words, the first cut 17 and the second cut 19 when combined provide a full cut or break of the moving web 12. The leading portion 14 and trailing portion 16 of the moving web 12 remains bridged together by the fastener assembly 18. The second cut 19 can include a perforation, die cut or other series of cuts, or breakage or separation of materials.

The second cut 19 may extend across the entire cross-direction width of the moving web 12, or along only a portion thereof. In some embodiments, the second cut 19 can be tapered. In addition, the second cut 19 can also be curved. The second cut 19 preferably has a length and which may or may not have a defined width, and can include linear and non-linear patterns, such as curvilinear patterns cuts, or other shapes, such as a circles, rectangles, etc. In other embodiments, the second cut 19 may be a series of cuts removing a portion of the fastener and moving web to form shaped cuts. The shaped cuts may be circles, squares, or any other shape known to one skilled in the art.

In a desirable embodiment, the second cutting station 40 forms a series of intermittent cuts in the moving web 12. In one desirable embodiment, second cut 19 includes a series of intermittent cuts, each perforation extending in both a machine direction and a cross-machine direction across the moving web 12. In another embodiment, the second cut 19 includes a series of intermittent cuts extending in only a machine direction or a cross-machine direction across the moving web 12. In other embodiments, the second cut 19 includes at least two lines of intermittent cuts, wherein the two lines of intermittent cuts when combined extend across the entire cross-machine direction of the moving web 12 and extend in at least one overlapping area in the machine direction. In other embodiments, the second cut 19 may be removing the outer edges of the moving web 12. The second cut 19 may be any other shape of cut known to those skilled in the art as long as the second cut 19 intersects with the first cut 17 and completely separates between the leading portion 14 and the trailing portion 16 of the moving web 12.

In one desirable embodiment, the second cutting station 40 is configured as a die cutter. Alternatively, the second cutting station 40 can be configured with a knife and anvil roll, laser, water jet, or other types of cutters known to those skilled in the art. In other alternative embodiments, the second cutting station 40 can comprise a device for applying heat, thermal energy or ultrasonic energy to the moving web 12 and fastener assembly so as to create a second cut 19 through both the moving web 12 and fastener assembly 18 at specific locations. In other preferred embodiments, the second cutting station 40 can include a chemical applicator that applies various chemicals, including for example water, to the web and fastener assembly to break it at specific locations. In yet another alternative embodiment, the apparatus applies a speed differential to the moving web 12 and the fastener assembly 18 so as to form a second cut 19 through both the moving web 12 and fastener assembly 18. Of course, it should be understood that the second cutting station 40 can also be configured from combinations of one or more of the above-referenced devices.

Optionally, the process includes monitoring the attachment of the fastener assembly 18. If the fastener assembly 18 is properly attached, the second cut 19 is completed. If the fastener assembly 18 is not properly attached, the second cut 19 is not completed. Desirably, the different stations may be formed from a single apparatus or different apparatuses depending on the setup of the machine.

FIGS. 3A-C through FIGS. 9A-9C depict exemplary embodiments for patterns for a first cut and a second cut that when combined will intersect and provide a fully severed or cut moving web 12. These figures only illustrate the moving web and not the fastener assembly. As described above, the second cut would also occur through the fastener assembly. Of course, these examples are not exhaustive and only illustrate a few of the possible patterns. One skilled in the art may utilize any other combination of patterns for the first cut and second cut that would provide a full cut across the moving web 12 to fully sever the moving web 12. For each of these embodiments, a cut extending in the machine direction extends in the transverse direction on the garment while a cut extending in the cross-machine direction extends in the longitudinal direction on the garment.

For example, FIG. 3A-C illustrates a first pattern combination for producing a fully severed moving web 12 with a first cut 30 and a second cut 32. FIG. 3A illustrates a first cut 30 formed from a series of intermittent cuts 31 extending in only the cross-machine direction. Similarly, FIG. 3B illustrates a second cut 32 formed from a series of intermittent cuts 33 extending in only the cross-machine direction. As illustrated in the Figures, the series of intermittent cuts 33 of the second cut 32 intersects with the series of intermittent cuts 31 of the first cut 30 resulting in full cut 35 across the entire moving web 12. In this embodiment, the second cut 32 would need to be registered with the first cut 30 to enable precise location of the second cut 32 and ensure full breakage of the moving web 12.

In another example, FIG. 4A-4C illustrates a second pattern combination for producing a fully severed moving web 12 with a first cut 40 and a second cut 42. FIG. 4A illustrates a first cut 40 formed from a series of intermittent cuts 41 extending in only the cross-machine direction. FIG. 4B illustrates a second cut 42 formed from a series of intermittent cuts 43 extending in both the machine direction and the cross-machine direction. In particular, this embodiment illustrates the series of intermittent cuts 43 cut as a half-circle. As illustrated in the Figures, the series of intermittent cuts 43 of the second cut 42 intersects with the series of intermittent cuts 41 of the first cut 40 resulting in full cut 45 across the entire moving web 12. In this embodiment, the second cut 42 may be registered with the first cut 40 to enable precise location of the second cut 42 and ensure full breakage of the moving web 12. However, since the location of the second cut 42 does not have to be precisely located since it extends in both the both the machine direction and the cross-machine direction, the second cut would not need to be precisely registered with the first cut 40 and still provide a full cut 45 through the moving web 12.

In another example, FIG. 5A-5C illustrates an alternative pattern combination for producing a fully severed moving web 12 with a first cut 50 and a second cut 52. FIG. 4A illustrates a first cut 50 formed from a series of intermittent cuts 51 extending in both the machine direction and the cross-machine direction. FIG. 4B illustrates a second cut 52 formed from a series of intermittent cuts 53 extending in both the machine direction and the cross-machine direction. As illustrated in the Figures, the series of intermittent cuts 53 of the second cut 52 intersects with the series of intermittent cuts 51 of the first cut 50 resulting in full cut 55 across the entire moving web 12.

In another example, FIG. 6A-6C illustrates a second pattern combination for producing a fully severed moving web 12 with a first cut 60 and a second cut 62. FIG. 6A illustrates a first cut 60 formed from at least two lines of intermittent cuts 61. As illustrated, at least a portion of the intermittent cuts 61 in the two lines extend across the entire cross-machine direction of the moving web without completely breaking the web. The intermittent cuts 61 additionally extend in at least one overlapping area 66 in the machine direction. FIG. 6B illustrates a second cut 62 formed from a series of intermittent cuts 63 extending in the machine direction. As illustrated in the Figures, the series of intermittent cuts 63 of the second cut 62 intersects with the series of intermittent cuts 61 of the first cut 60 in the overlapping area 66 resulting in full cut 65 across the entire moving web 12.

In another example, FIG. 7A-7C illustrates a second pattern combination for producing a fully severed moving web 12 with a first cut 70 and a second cut 72. FIG. 7A illustrates a first cut 70 formed a single cut extending in the cross-machine direction of the moving web 12 without completely breaking the web leaving outer edges 76, 77 of the moving web 12 intact. FIG. 7B illustrates a second cut 72 involves a cut-out of the outer edges of the moving web 12. As illustrated in the figures, the second cut 72 intersects with the first cut 70 resulting in full cut 75 across the entire moving web 12.

In another example, FIG. 8A-8C illustrates another pattern combination for producing a fully severed moving web 12 with a first cut 80 and a second cut 82. FIG. 8A illustrates a first cut 80 formed from a series of intermittent cuts 81 extending in only the cross-machine direction. FIG. 8B illustrates a second cut 82 formed from a series of shaped cuts 83 removing a portion of both the moving web 12 and fastener assembly. As illustrated in the Figures, the series of intermittent cuts 83 of the second cut 82 intersects with the series of intermittent cuts 81 of the first cut 80 resulting in full cut 85 across the entire moving web 12.

In another embodiment, FIG. 9A-9C illustrates another pattern combination for producing a substantially severed moving web 12 with a first cut 86 and a second cut 88. FIG. 9A illustrates a first cut 86 formed from a series of intermittent cuts 87. FIG. 9B illustrates a second cut 88 formed from a series of intermittent cuts 89 through both the moving web 12 and fastener assembly. As illustrated in the Figures, the series of intermittent cuts 83 of the second cut 82 intersects with the series of intermittent cuts 81 of the first cut 90 resulting in a cut 90 that substantially separates the moving web 12. In other words, the cut 90 does not provide a cut across the entire moving web, but maintains some connection to between the leading portion and trailing portion of the moving web. To be substantially separated, at least 90% of the moving web between a waist end and leg cut-out end has been cut.

In another example, FIG. 10A-10C illustrates another embodiment for producing a fully severed moving web 12 with a first cut 91 and a second cut 93. FIG. 10A illustrates a first cut 91 formed from a series of intermittent cuts 92 extending in only the cross-machine direction. FIG. 10B illustrates a fastener assembly 18 with a series of holes formed therethough 95 and a second cut 93 formed from a series of intermittent cuts 94 cutting through a portion of the moving web 12. The second cut 93 is formed through the holes 95 in the fastener assembly 18 without cutting through the fastener assembly 18 and only cutting the moving web 12. As illustrated in the Figures, the series of intermittent cuts 94 of the second cut 93 intersects with the series of intermittent cuts 92 of the first cut 91 resulting in full cut 96 across the entire moving web 12.

Various methods and apparatus for manufacturing absorbent garments and for applying fastener members thereto are disclosed in U.S. Pat. No. 6,730,188 entitled “Method and Apparatus For Assembling Refastenable Absorbent Garments,” U.S. Pat. No. 6,743,321 entitled “Method and Apparatus For Assembling Refastenable Absorbent Garments,” U.S. Pat. No. 6,686,626 entitled “Method and Apparatus For Assembling Refastenable Absorbent Garments,” U.S. Pat. No. 6,682,626 entitled “Method and Apparatus For Assembling Refastenable Absorbent Garments,” U.S. Pat. No. 6,712,922 entitled “Multiple Component Web,” U.S. Pat. No. 6,730,188 entitled “Method of Assembling Personal Care Absorbent Article,” U.S. Pat. No. 6,783,487, entitled “Pant-Type Personal Care Articles, and Methods of Making and Using Such Personal Care Articles,” U.S. Pat. No. 6,454,888 entitled “Methods of Changing Size of Pant-Type Personal Care Articles Outputted from a Manufacturing Process,” the entire disclosures of which are hereby incorporated by reference.

In one embodiment, illustrated in FIG. 1, at a fourth position A4, the moving web 12 is used as a front body panel web and bonded to a rear body panel web 28 at side seams, wherein the rear body panel web 28 is positioned over the moving web 12 by folding a crotch portion 29, joining or bridging between the moving web 12 and the back body panel web 28.

Generally, the method described above produces a pre-fastened adjustable pant-like disposable absorbent undergarment having a body panel and a fastener assembly attached to the body panel, wherein at least one side of the fastener assembly is releasably attached to the body panel. The body panel contains a first cut through the body panel under the fastener assembly defining a first section of the body panel and a second section of the body panel. A second cut through both the body panel and fastener assembly intersects the first cut to completely separate the first section of the body panel and the second section of the body panel, wherein the first section and second section remains bridged together by the fastener assembly.

FIGS. 11-13 illustrate one embodiment of an adjustable pant-like disposable absorbent undergarment 100 having a longitudinal axis 111 made using the method described herein. The adjustable pant-like disposable absorbent undergarment 100 is designed to absorb liquid, semi-solid and/or solid waste discharged from a human being. The adjustable pant-like disposable absorbent undergarment 100 is designed to absorb and/or retain one or more bodily discharges of waste material such as urine, perspiration, excrement, feces, menses, menstrual fluid, as well as other liquid and/or solid waste.

The adjustable pant-like disposable absorbent undergarment 100 includes a front body panel 112, a back body panel 114 and an absorbent assembly 116 secured to the front and back body panels, 112 and 114 respectively. The front and back body panels, 112 and 114 respectively, are joined together by a pair of seams 118 and 120 to form a waist opening 122 and a pair of leg openings 124 and 126.

The front body panel 112 includes a waist edge 128, a crotch edge 130 and a pair of side edges 132 and 134. In a three piece construction wherein an absorbent assembly 116 is secured between the front body panel 112 and the back body panel 114, the crotch edge 130 is well defined. In absorbent undergarments of a different construction, the crotch edge 130 can be an imaginary line transversely drawn between the pair of leg openings 124 and 126 at a location where one considers the front body panel 112 to end. The exact size and configuration of the front body panel 112 can vary to suit a wearer's particular needs.

As illustrated, the front body panel is separated into multiple sections, the multiple sections bridged together with a fastener assembly at each separation. For example, the front body panel 112 may have a first side section 136, a middle section 138 and a second side section 140. The front body panel 112 is completely severed between both the first side section 136 and the middle section 138 and the second side section 140 and the middle section 138 creating a first fully severed region, or gap, 142 and a second fully severed region 144. The first side section 136 is aligned adjacent to the side edge 132 at its leading edge forming the seam 120 and the second side section 140 is aligned adjacent to the side edge 134 at its leading edge forming the seam 118. The middle section 138 has a first edge 132 and a second edge 134 and is located between the first and second sections, 136 and 140 respectively. The terminal edge 150 of the first side section 136 is aligned adjacent to the first edge 152 of the middle section 138 and the terminal edge 150 of the second side section 140 is aligned adjacent to the second edge 154 of the middle section 138. As depicted, the middle section 138 is centrally located and is bifurcated by the longitudinal axis 111 at a midpoint of the front body panel 112.

The first and second fully severed regions, 142 and 144, can be linear or non-linear in configuration. In FIGS. 11-13, the first and second fully severed region, 142 and 144, are shown having a linear or straight configuration. The first and second fully severed regions, 142 and 144, extend longitudinally from approximately the waist edge 128 down to approximately one of the leg openings 124 or 126. The first and second fully severed regions, 142 and 144, can be aligned parallel to the longitudinal axis 111 or be angled thereto. For example, the first and second fully severed regions, 142 and 144, can be tapered relative to the longitudinal axis 111, if desired. In addition, the fully severed regions, 142 and 144, can also be curved. The shapes of the first and second fully severed regions, 142 and 144, may match the patterns found in FIGS. 3-8.

Still referring to FIGS. 11-13, each of the first and second fully severed regions, 142 and 144, extend from approximately the waist edge 128 of the front body panel 112 to one of the pair of leg openings 124 and 126. Another way of describing this is to say that the first and second fully severed regions, 142 and 144, extend from approximately the waist edge 128 of the front body panel 112 to approximately the crotch edge 130 of the front body panel 112.

Many adjustable, pant-like disposable absorbent undergarments include a pair of lines of weakness to break to allow for the individual to adjust the size of the pants. This type of adjustable disposable undergarment including the lines of weakness requires an amount of force needed to break the lines of weakness. The pre-fastened adjustable, pant-like disposable absorbent undergarments 100 described herein have the first and second fully severed regions, 142 and 144, that require no force to break allowing for easier use by an elderly individual, caretaker or parent caring for a child.

Still referring to FIGS. 11-13, each of the first and second fully severed regions, 142 and 144, is shown being aligned parallel to one of the pair of seams 118 and 120. Such an arrangement provides for an aesthetically pleasing appearance to the front body panel 112.

Desirably, in the pre-fastened condition, the first side sections 136 and the middle section 138 of front body panel 112 do not overlap, and the second side section 140 and the middle section 138 of the front body panel 112 do not overlap in the severed regions 142, 144. In one exemplary embodiment, a first gap may be formed in the first severed region 142 between the first side section 136 and the middle section 138 of the front body panel 112 in the pre-fastened condition. In this embodiment, a second gap may be formed in the first severed region 142 between the second side section 140 and the middle section 138 of the front body panel 112 in the pre-fastened condition. In another exemplary embodiment, a terminal or outer edge 150 of the first side section 136 abuts against the first edge 152 of the middle section 138 of front body panel 112 in the pre-fastened condition. In this embodiment, a terminal or outer edge 150 of the second side section 140 abuts against the second edge 154 of the middle section 138 of the front body panel 112 in the pre-fastened condition. In another embodiment, a gap may be formed between one side section and the middle section 138 and the other side section and middle section 138 abut against each other. If a gap is formed between the side sections 136 and 140, and the middle section 138, the gap will desirably be less than 10 mm.

Referring again to FIGS. 11-13, the front body panel 112 further includes a pair of fastener assemblies 168 and 170. Each fastener assembly 168 and 170 includes a first portion 172 and a second portion 174. The first portion 172 of the fastener assembly 168 can be permanently secured to the first side panel 136 of the front body panel 112 and the first portion 172 of the other fastener assembly 170 can be permanently secured to the second side panel 140 of the front body panel 112. As illustrated, each fastener assembly 168, 170 has a cut 171 formed therethrough.

In the embodiment shown in FIGS. 11-13, the first portions 172 of the fastener assemblies 168 and 170 can be securely attached using an adhesive, heat, pressure, a combination of heat and pressure, an ultrasonic bond, a chemical bond or by other means known to those skilled in the art.

Each of the second portions 174 of the fastener assemblies 168 and 170 are depicted as extending almost the entire length of the fully severed portions 142 and 144. This means at least 50% of the distance between the waist edge 128 and the leg opening 124, 126 are covered by the fastener assembly 18. Desirably, the first fastener assembly 168 and second fastener assembly 170 extend at least 65% of the distance between the waist edge 128 and the leg opening 124, 126. More desirably, the first fastener assembly 168 and second fastener assembly 170 extend at least 75% of the distance between the waist edge 128 and the leg opening 124, 126. Even more desirably, the first fastener assembly 168 and second fastener assembly 170 extend at least 85% of the distance between the waist end and the leg opening. Even more desirably, the first fastener assembly and second fastener assembly completely extend at least 95% of the distance between the waist edge 128 and the leg opening 124, 126. The second portions 174 of the fastener assembly 168 can bridge across the first fully severed region 142 and the second portion 174 of the other fastener assembly 170 can bridge across the second fully severed region 144. By extending substantially the entire length of and bridging the fully severed portions, 142 and 144, the fasteners 168, 170 cover the fully severed portions 142, 144 and provide a more underwear like appearance. The second portions 174 of the fastener assemblies 168 and 170 can be releasably attached to the middle section 138 of the front body panel 112.

Alternatively, it should be evident to those skilled in the art that the first portion 172 of each of the fastener assemblies 168 and 170 could be permanently attached to the middle section 138. In this embodiment, the second portions 174 of the fastener assemblies 168 and 170 can be releasably attached to the side sections of the front body panel 112. In an alternative embodiment, both the first portion 172 and the second portion 174 may be releasably attached. The fastener assembly 168, 170 may be constructed of a non-extensible, stretchable, or elastomeric material.

Referring now to FIGS. 12 and 13, each of the second portions 174 of the fastener assemblies 168 and 170 has an inner surface 180 that contains a fastener 182. The fastener 182 can be a mechanical fastener 182. In FIGS. 13 and 13, the mechanical fastener 182 is shown as a plurality of fine hooks, such as VELCRO hooks. VELCRO is a trademark of Velcro USA, Inc. (Manchester, N.H.). The hooks are designed to easily engage and be removed from a material wherein the material has a loose weave pattern or the fibers forming the material will allow the hooks to be attached to them. The mating material is commonly referred to as the loop member of a hook and loop fastener. The middle section 138 of the front body panel 112 may be formed of such a material. When the hooks engage into the middle section 138, a secure but releasable fastener is formed. The hooks can be easily removed from the loop material by pulling the edge of the fastener outward away from the middle section 138. Alternatively, as illustrated in FIGS. 12 and 13, a fastening component 183 such as a loop material or loose fibers that may be placed anywhere onto the middle section 138 to facilitate the fastening component. Therefore, hook and loop fasteners are referred to as being releasable and can be fastened and released several times. In alternative embodiments, the mechanical fastener 182 constructed of hooks may be placed on to the middle section 138 of the front body panel 112. In this embodiment, the fastening component 183 constructed of a loop material or loose fibers would be placed on the fastener assemblies 168, 170. Both the mechanical fastener 182 and the fastening component 183 may be integral or separately attached.

Referring again to FIGS. 11-13, one can see that FIG. 11 depicts the pair of fastener assemblies 168 and 170 being securely fastened to the middle section 138 of the front body panel 112. If the wearer of the disposable absorbent undergarment 100 or a caregiver wishes to inspect the undergarment 100, he or she would open the pair of fastener assemblies 168 and 170 to the position shown in FIG. 12. Since the first and fully severed portions, 142 and 144 are already broken, the middle section 138 of the disposable absorbent undergarment 100 can be easily moved outward away from the wearer's torso. The wearer can then inspect the absorbent assembly 116 to see if it needs to be changed. If so, the disposable absorbent undergarment 100 can be removed from about the wearer's torso and be replaced by another undergarment. If the absorbent assembly 116 is still capable of accepting additional body fluid, the middle section 138 is moved back against the wearer's torso and the pair of fastener assemblies 168 and 170 is refastened to the middle section 138.

The horizontal distance to each of the first and second fully severed regions, 142 and 144, and corresponding first and second fastener assemblies, 168 and 170, as located relative to the pair of side seams 118 and 120 can also vary. A distance “D” is depicted in FIGS. 11-13 which represents the distance between each of the pair of seams 118 and 120 and the corresponding first and second fully severed regions, 142 and 144 respectively. One can increase the distance “D” by moving the first and second fully severed regions, 142 and 144 respectively, away from the pair of seams 118 and 120. When one increases the distance “D”, one may find that it is easier for the wearer of the disposable absorbent undergarment 100 to visually see and identify the first and second fully severed regions, 142 and 144, when user looks down at the front body panel 112. In some embodiments, the first fastener assembly 168 and the second fastener assembly 170 are located near the midpoint between the side seams, 118 and 120, and the cross-sectional midpoint 111 of the front body panel 112. Desirably, the first fastener assembly 68 and second fastener assembly 70 are located between 30% and 70% of the distance between the side seam, 118 and 120, and the cross-sectional midpoint 111 of the front panel 112. More desirably, the first fastener assembly 168 and second fastener assembly 170 are located between 35% and 45% of the distance between the side seams, 118 and 120, and the cross-sectional midpoint 111 of the front panel 112. A manufacturer is free to vary the distance “D” to best suit the size and shape of a particular disposable absorbent undergarment 100 to make sure that the fastener is on the front of the product rather than the side of the product. If the fully severed regions are not parallel to the side seam, the midpoint of the fully severed regions in the longitudinal direction may be used to measure “D”.

Placement near the midpoint between the side seam 118 and the cross-sectional midpoint 111 of the front body panel 112 facilitates enhanced donning and removal when experiencing a reduced range of motion as well as locating the fastener in a location which is easier for the consumer to see.

The placement of the fastener assemblies 168 and 170 in the front of the product optimizes the ability of the user to fasten the product, while minimizing potential pop-opens during use due to movement of the legs of the user. The placement of the fasteners can be measured in both an unstretched (out of the bag) state or stretched (as in use).

It should be understood that various other embodiments, modifications, and equivalents to the embodiments of the absorbent article described herein which, after reading the description herein, may suggest themselves to those skilled in the art without departing from the scope and spirit of the present claims.

What is claimed is:

1. A method of producing pre-fastened disposable garments, comprising:
a. providing a moving web for use as a body panel;
b. cutting a first cut through the moving web to define a leading portion of the moving web and a trailing portion of the moving web connected at the first cut;
c. bridging the connected leading portion of the moving web and trailing portion of the moving web together with a fastener assembly that extends over the first cut; and
d. cutting a second cut through both the moving web and fastener assembly intersecting the first cut such that the leading portion of the moving web and trailing portion of the moving web completely separate, wherein the leading portion and trailing portion remains bridged together by the fastener assembly.

2. The method of claim 1 wherein the first cut comprises a series of intermittent cuts extending in both a machine direction and a cross-machine direction.

3. The method of claim 2 wherein the second cut comprises a series of cuts extending in both the machine direction and the cross-machine direction.

4. The method of claim 2 wherein the second cut comprises a series of cuts extending in only the machine direction or the cross-machine direction.

5. The method of claim 1 wherein the first cut comprises a series of cuts extending in only a cross-machine direction.

6. The method of claim 4 wherein the second cut comprises a series of cuts extending in both a machine direction and the cross-machine direction.

7. The method of claim 1 wherein the second cut comprises a series of cuts removing a portion of the fastener and moving web to form shaped cuts.

8. The method of claim 1 wherein the first cut comprises at least two lines of intermittent cuts, wherein the two lines of intermittent cuts extend across the entire cross-machine direction of the moving web and extend in at least one overlapping area in the machine direction; and
the second cut comprises at least one cut extending in the machine direction and intersects the two lines of intermittent cuts in the overlapping area.

9. The method of claim 1 wherein the first cut comprises a cut extending in the cross-machine direction of the moving web leaving an edge portion of each edge of the moving web attached and the second cut comprises removing the edge portions of each edge of the moving web.

10. A pre-fastened adjustable pant-like disposable absorbent undergarment comprising:
a. a body panel;
b. a fastener assembly attached to the body panel, wherein at least one side of the fastener assembly is releasably attached to the body panel;
c. a fully severed body panel under the fastener assembly defining a first section of the body panel and a second section of the body panel; and
d. wherein the fastener assembly has at least one cut therethrough.

11. The pre-fastened adjustable pant-like disposable absorbent undergarment of claim 10 comprising a first cut through the body panel under the fastener assembly defining a first section of the body panel and a second section of the body panel; and
a second cut through the body panel and fastener assembly intersecting the first cut such that the first section of the body panel and the second section of the body panel completely separate, wherein the first section and the second section remains bridged together by the fastener assembly.

12. The pre-fastened adjustable pant-like disposable absorbent undergarment of claim 11 wherein the first cut comprises a series of intermittent cuts extending in both a transverse direction and a longitudinal direction.

13. The pre-fastened adjustable pant-like disposable absorbent undergarment of claim 12 wherein the second cut comprises a series of cuts extending in both the transverse direction and the longitudinal direction.

14. The pre-fastened adjustable pant-like disposable absorbent undergarment of claim 12 wherein the second cut comprises a series of cuts extending in only the transverse direction or the longitudinal direction.

15. The pre-fastened adjustable pant-like disposable absorbent undergarment of claim 11 wherein the first cut comprises a series of cuts extending in only a longitudinal direction.

16. The pre-fastened adjustable pant-like disposable absorbent undergarment of claim 15 wherein the second cut comprises a series of cuts extending in both a transverse direction and the longitudinal direction.

17. The pre-fastened adjustable pant-like disposable absorbent undergarment of claim 11 wherein the first cut or second cut comprises at least two lines of intermittent cuts, wherein the two lines of intermittent cuts when combined extend across the entire longitudinal direction and extend in at least one overlapping area in the transverse direction; and
the second cut comprises at least one cut extending in the transverse direction and intersects the two lines of intermittent cuts in the overlapping area.

18. The pre-fastened adjustable pant-like disposable absorbent undergarment of claim 11 wherein the first cut comprises a cut extending in the longitudinal direction leaving a waist edge portion and a crotch edge portion on the body panel attached and the second cut comprises removing the waist edge portion and a crotch edge portion of the body panel.

19. A method of producing pre-fastened disposable garments, comprising:
a. providing a moving web for use as a body panel;
b. cutting a first cut through the moving web to define a leading portion of the moving web and a trailing portion of the moving web connected at the first cut;
c. bridging the connected leading portion of the moving web and trailing portion of the moving web together with a fastener assembly that extends over the first cut; and
d. monitoring attachment of the fastener assembly.

20. The method of claim 19 wherein monitoring attachment of the fastener assembly involves observing attachment of the fastener assembly, the method further comprising cutting a second cut through both the moving web and fastener assembly intersecting the first cut such that the leading portion of the moving web and trailing portion of the moving web completely separate, wherein the leading portion and trailing portion remains bridged together by the fastener assembly.

21. The method of claim 19 wherein monitoring attachment of the fastener assembly involves observing no attachment of the fastener assembly, the method further comprising not making a second cut of the moving web.

22. A method of producing pre-fastened disposable garments, comprising:
a. providing a moving web for use as a body panel having a waist end and a leg cut-out end;
b. cutting a first cut through the moving web to define a leading portion of the moving web and a trailing portion of the moving web connected at the first cut;
c. bridging the connected leading portion of the moving web and trailing portion of the moving web together with a fastener assembly that extends over the first cut; and
d. cutting a second cut through both the moving web and fastener assembly intersecting the first cut such that the leading portion of the moving web and trailing portion of the moving web substantially separates, wherein the leading portion and trailing portion remains bridged together by the fastener assembly.

23. The method of claim 21 wherein moving web substantially separating comprises at least 90% of a length of the moving web between the waist end and the leg cut-out end being cut.

24. A method of producing pre-fastened disposable garments, comprising:
a. providing a moving web for use as a body panel;
b. cutting a first cut through the moving web to define a leading portion of the moving web and a trailing portion of the moving web connected at the first cut;
c. bridging the connected leading portion of the moving web and trailing portion of the moving web together with a fastener assembly that extends over the first cut, the fastener assembly having at least one hole formed therethough; and
d. cutting a second cut through the moving web through the at least one hole in the fastener assembly, the second cut intersecting the first cut such that the leading portion of the moving web and trailing portion of the moving web completely separate, wherein the leading portion and trailing portion remains bridged together by the fastener assembly.
